(19) United States
(12) Patent Application Publication
(10) Pub. No.: US 2017/0042461 A1
(43) Pub. Date: Feb. 16, 2017

(54) TECHNIQUES TO EVALUATE AND ENHANCE COGNITIVE PERFORMANCE

(71) Applicant: BATTELLE MEMORIAL INSTITUTE, Richland, WA (US)

(72) Inventors: Nathan O. Hodas, West Richland, WA (US); Kristina Lerman, Los Angeles, CA (US); Lyndsey Franklin, West Richland, WA (US); Dustin Arendt, Richland, WA (US)

(73) Assignee: BATTELLE MEMORIAL INSTITUTE, Richland, WA (US)

(21) Appl. No.: 15/212,138

(22) Filed: Jul. 15, 2016

Related U.S. Application Data
(60) Provisional application No. 62/193,409, filed on Jul. 16, 2015.

(51) Int. Cl. A61B 5/16; G09B 7/00

(52) U.S. Cl. CPC A61B 5/16; G09B 7/00

(57) ABSTRACT

In one embodiment of the present application, an operator provides entries into the computer workstation indicative of an evaluation. Assessment of the entries takes place relative to data representative of a corresponding multiple resource neuroenergetic model for the cognitively demanding activity. Generation of an operator instruction results from the assessment that is provided to the operator through the computer workstation. The operator instruction directs the operator to take a break from the cognitively demanding activity based on the assessment.

### CROSS-REFERENCE TO RELATED PATENT APPLICATIONS

The present application claims the benefit of U.S. Provisional Patent Application No. 62/193,409 filed 16 Jul. 2015, which is hereby incorporated by reference herein in its entirety.

### STATEMENT REGARDING RIGHTS TO INVENTION MADE UNDER FEDERALLY-SPONSORED RESEARCH AND DEVELOPMENT

This invention was made with Government support under Contract DE-AC05-76RLO1830 awarded by the U.S. Department of Energy. The Government has certain rights in the invention.

### BACKGROUND

The present application relates generally to unique techniques, systems, methods, processes, apparatus, combinations, and devices to address machine-aided cognitive enhancement, and more specifically, but not exclusively the evaluation of cognitive performance based on neuroenergetic modeling to identify unique ways to augment cognitive performance.

Standardized testing plays a central role in educational and vocational evaluation. By analyzing performance of a large cohort answering practice standardized test questions online, it has been observed that accuracy and learning decline as the test session progresses, but improve following a brief break. In general, modeling human data poses considerable challenges because of the large variation among subjects. Indeed, the evaluation of cognitive performance is persistently plagued by a wide degree of cognitive heterogeneity among test subjects. There have been several attempts to reduce such effects with, at most, limited success. While it may be casually observed that some can perform certain cognitive exercises with much greater proficiency than others based on the nature of the exercise, it is much more obscure what factors may account for differing exercise results—such as background of the individual subject to testing like education level, academic performance, and proper test-taking habits, just to name a few examples. There has been limited success in eliminating factors that mask the evaluation of certain core cognitive/neurological mechanisms that may account for underperformance of the population generally. Among other things, distancing the impact of heterogeneous disparities from such core mechanisms can aid in the strengthening of various standardized measures of evaluation.

It has been proposed that certain testing causes some form of degradation over time that limits ability to maintain the speed and/or accuracy observed during earlier stages of testing. It is hypothesized that answering questions consumes some finite cognitive resource(s). Indeed, one growing line of inquiry suggests that human decision making, performance on visual attention tasks, self-control, and even morality may become more limited over time. One theory suggests that the energetic resources of the human body needed to perform mental work diminish over time and usage, and performance correspondingly declines. Some studies have linked impulsive behavior, the loss of willpower, and executive function (so-called “ego depletion”) with consumptive depletion; however, these suppositions have been controversial, and there are numerous competing, alternative hypotheses. The controversy stems in part from the difficulties of measuring ego depletion and replicating its effects in laboratory experiments; and further from the lack of clear mechanisms to characterize the depletion process. Thus, there remains a need for further contributions in this area of technology.

By way of transition from this Background to subsequent sections of the present application, one or more abbreviations, acronyms, and/or definitions are set forth below and supplemented by example or further explanation where deemed appropriate. Among other things, these definitions are provided to: (a) resolve meaning sometimes subject to ambiguity and/or dispute in the applicable technical field(s) and/or (b) exercise the lexicographic discretion of any named inventor(s), as applicable:
1. “α-KG is an acronym for α-Ketoglutaric acid that is one of two ketone derivatives of glutaric acid (defined below).
2. “Ac-CoA” refers to “Acryl-CoA” that is a group of coenzymes involved in the metabolism of fatty acids. Generally, it is a temporary compound formed when coenzyme A (CoA) attaches to the end of a long-chain fatty acid inside living cells. The compound undergoes beta oxidation, forming one or more molecules of acetyl-CoA. This, in turn, enters the citric acid cycle, eventually forming several molecules of ATP.
3. “ASP” is an abbreviation of “Aspartate” or “Aspartic Acid.” Aspartate is a common amino acid neurotransmitter.
4. “GLC” is an abbreviation of Glucose that is a sugar with the molecular formula C₆H₁₂O₆.
5. “GLN” is an abbreviation of Glutamine (also abbreviated as Gln or Q, and often called L-glutamine). It is one of the 20 amino acids encoded by the standard genetic code and is considered a conditionally essential amino acid. Its side-chain is an amide formed by replacing the side-chain hydroxyl of glutamic acid with an amine functional group, making it the amide of glutamic acid.
6. “GLU” is an abbreviation of Glutamatic Acid, which is one of the proteinogenic amino acids and its codons are GAA and GAG. The carboxylate anions and salts of GLU may be referred to as glutamates.
7. “Glutaric Acid” is the organic compound with the formula C₃H₆(COOH)₂.
8. “Glycogen” refers to a multi-branched polysaccharide of glucose that serves as a form of energy storage.
9. “Glycolysis” refers to the metabolic pathway that converts glucose C₆H₁₂O₆, into pyruvate, CH₃COCOO⁻+H⁺. The free energy released in this process is used to form the high-energy compounds ATP (adenosine triphosphate) and NADH (reduced nicotinamide adenine dinucleotide.
10. “LAC” is an abbreviation of Lactate or Lactic acid both of which play a role in various biochemical processes. Lactic acid is a carboxylic acid with the chemical formula C₂H₄OHCOOH. It has a hydroxyl group adjacent to the carboxyl group, making it an alpha hydroxy acid (AHA). In solution, it can lose a proton from the carboxyl group, producing the lactate ion CH₃CH(OH)COO⁻.
11. “NNA” is an abbreviation of N-Acetylaspartic acid, or N-acetylaspartate (NAA), is a derivative of aspartic acid with a formula of C₆H₉NO₅. NAA is the second-most-concentrated molecule in the brain after the amino acid glutamate. It is detected in the adult brains only in neurons and is synthesized in the mitochondria from the amino acid aspartic acid and acetyl-coenzyme A.
12. “OAA” is an abbreviation of oxaloacetate. Pyruvate carboxylase (PC) is an enzyme of the ligase class that catalyzes the irreversible carboxylation of pyruvate to form OAA.
13. “PDC” is an abbreviation of pyruvate dehydrogenase complex that is a complex of three enzymes that convert pyruvate into acetyl-CoA by a process called pyruvate decarboxylation. Acetyl-CoA may then be used in the citric acid cycle to carry out cellular respiration, and this complex links the glycolysis metabolic pathway to the citric acid cycle. Pyruvate decarboxylation is also known as the “pyruvate dehydrogenase reaction” because it also involves the oxidation of pyruvate.
14. “PYR” is an abbreviation of “Pyruvic” acid (CH₃COCOOH) is the simplest of the alpha-keto acids, with a carboxylic acid and a ketone functional group and sometimes is an abbreviation of “Pyruvate,” the conjugate base, CH₃COCOO⁻.
15. “TCA Cycle” refers to the TriCarboxylic Acid” cycle, the Krebs cycle, or sometimes the citric acid cycle. It is a series of chemical reactions used by aerobic organisms to generate energy through the oxidation of acetate derived from carbohydrates, fats and proteins into carbon dioxide and chemical energy in the form of adenosine triphosphate (ATP). In addition, the cycle provides precursors of certain amino acids as well as the reducing agent NADH that is used in numerous other biochemical reactions.
   The above listing of one or more abbreviations, acronyms, and/or definitions apply to any reference to the corresponding subject terminology herein unless explicitly set forth to the contrary. Any acronym, abbreviation, or terminology defined in parentheses, quotation marks, or the like elsewhere in the present application likewise shall have the meaning imparted thereby throughout the present application unless expressly stated to the contrary or unless identical to an entry of the immediately preceding numerical listing of abbreviations, acronyms, and/or definitions, in which case such listing prevails. Any acronym, abbreviation, or definition provided herein applies irrespective of whether the abbreviated, defined and/or otherwise represented terminology is in lower case, upper case, or capitalized form, unless expressly stated to the contrary.

### SUMMARY

Among the embodiments of the present application are unique systems, apparatus, methods, processes, combinations, and devices for the monitoring, enhancement, analysis, and/or application of cognitively demanding activities/tasks including the evaluation of variable visual information and assessment of the same relative to data representative of neuroenergetic modeling. Other embodiments include unique techniques to monitor, analyze, evaluate, enhance, apply, and augment cognitively intensive and/or demanding tasks.

Another nonlimiting embodiment of the present application comprises: receiving entries from an operator corresponding to evaluation of visual information in the performance of a cognitively demanding activity; analyzing the entries relative to data representative of a corresponding neuroenergetic model; generating an operator instruction in response to the analyzing of the entries; and directing a break period for the operator from the cognitively demanding activity in accordance with the operator instruction.

Yet another embodiment comprises equipment including: (a) means for receiving entries from an operator that corresponds to evaluation of visual information in the performance of a cognitively demanding activity; (b) means for analyzing the entries relative to data representative of a corresponding neuroenergetic model; (c) means for generating an operator instruction in response to the analyzing means; and (d) means for directing a break period for the operator from the cognitively demanding activity in accordance with the operator instruction.

The above introduction is not to be considered exhaustive or exclusive in nature—merely serving as a forward to further advantages, apparatus, applications, arrangements, aspects, attributes, benefits, characterizations, combinations, components, compositions, compounds, conditions, configurations, constituents, designs, details, determinations, devices, discoveries, elements, embodiments, examples, exchanges, experiments, explanations, expressions, factors, features, forms, formulae, gains, implementations, innovations, kits, layouts, machinery, materials, mechanisms, methods, modes, models, objects, options, operations, parts, processes, properties, qualities, refinements, relationships, representations, species, structures, substitutions, systems, techniques, traits, uses, utilities, and/or variations that shall become apparent from the description provided herewith, from any claimed invention, drawing, and/or other information included herein.

### BRIEF DESCRIPTION OF THE DRAWING(S)

For any figure introduced in the following description, like reference numerals refer to like features previously described.

FIG. 1 is a partially schematic view of a cognitive performance monitoring, evaluation, training, application, improvement, and enhancement system of one embodiment of the present application.

FIG. 2 is a flow chart of a routine to evaluate cognitive performance based on a statistically significant pool of test subjects utilizing the system of FIG. 1.

FIG. 3 is a comparative graph of relative cognitive performance based on 50 initial questions (normalized value on vertical axis) versus logarithmic units of time in minutes spent answering these questions without a break (horizontal axis).

FIGS. 4-7 are graphic views demonstrating observed relationships between performance characteristics (vertical axes) and time (horizontal axes). FIG. 4 is a graph of normalized performance approaching a five (5) minute break (vertical axis) versus logarithmic units of time in minutes (horizontal axis) five minutes before such break. FIG. 5 is a graph of relative answer speed approaching a break (normalized value on vertical axis) versus logarithmic units of time in minutes before the break (horizontal axis). FIG. 6 is a graph of percentage (%) for which a repeated question is answered correctly (vertical axis) versus logarithmic units of time in minutes before a break (horizontal axis). FIG. 7 is a graph of change in normalized performance (vertical axis) versus logarithmic time in units of minutes for length of a break (horizontal axis).

FIG. 8 is a partial diagrammatic view representative of selected brain anatomy and biochemistry pertinent to certain neuroenergetic modeling of cognitive brain stimulation in correspondence to observations in FIGS. 3-7 and FIGS. 9-15.

FIGS. 9-15 are graphic views plotting selected observables versus Primary (P) and Secondary (S) sources according to a certain two-source neuroenergetic model. FIG. 9 is a graph of relative answer duration (normalized value on vertical axis) versus relative primary resource P usage (horizontal axis). FIG. 10 is a graph of relative answer duration (normalized value on vertical axis) versus relative secondary resource S usage (horizontal axis). FIG. 11 is a graph of learning probability (vertical axis) versus relative primary resource P usage (horizontal axis). FIG. 12 is a graph of learning probability (vertical axis) versus relative secondary resource S usage (horizontal axis). FIG. 13 is a graph of performance (vertical axis) versus relative primary resource P usage (horizontal axis). FIG. 14 is a graph of performance (vertical axis) versus relative secondary resource S usage (horizontal axis). FIG. 15 is a graph of mean difficulty (vertical axis) versus relative primary resource P usage (horizontal axis).

FIG. 16 is a flow chart of certain nonlimiting applications resulting from the investigation of neuroenergetic modelling as reflected in FIGS. 1-15.

### DETAILED DESCRIPTION OF REPRESENTATIVE EMBODIMENTS

In the following description, various details are set forth to provide a thorough understanding of the principles and subject matter of each invention described and/or claimed herein. To promote this understanding, the description refers to representative embodiments—using specific language to communicate the same accompanied by any drawing(s) to the extent the description subject matter admits to illustration. In other instances, when the description subject matter is well-known, such subject matter may not be described in detail and/or may not be illustrated by any drawing(s) to avoid obscuring information to be conveyed hereby.

Considering the invention(s) defined by the description and/or the claim(s) further, those skilled in the relevant art will recognize that in at least some instances such invention(s) can be practiced without one or more specific details included in the description. It is also recognized by those skilled in the relevant art that the full scope of an invention described and/or claimed herein can encompass more detail than that made explicit herein. Such unexpressed detail can be directed to apparatus, applications, arrangements, combinations, components, compositions, compounds, conditions, configurations, constituents, designs, devices, elements, embodiments, features, forms, formulae, implementations, kits, modifications, materials, mechanisms, methods, modes, operations, parts, processes, properties, qualities, refinements, relationships, structures, systems, techniques, and/or uses—just to name a few. Accordingly, this description of representative embodiments should be seen as illustrative only and not limiting the scope of any invention described and/or claimed herein.

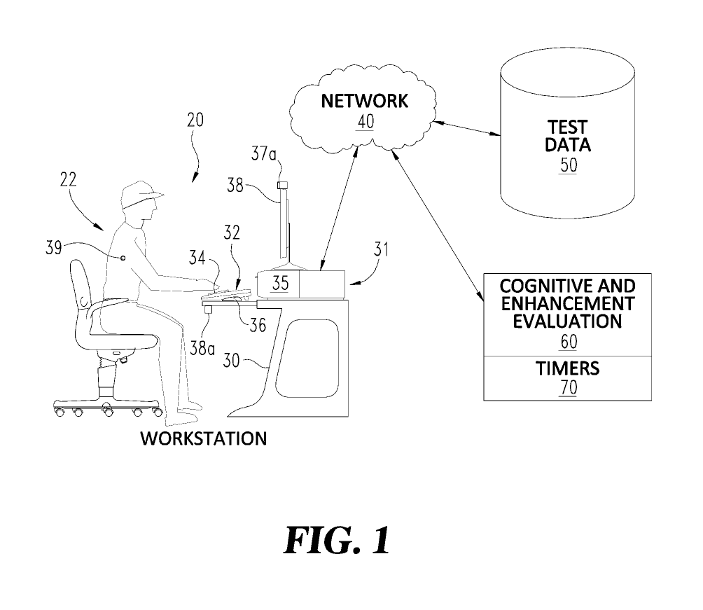

FIG. 1 is a view of a partially diagrammatic system 20 suitable for cognitive performance evaluation, monitoring, training, enhancement, and various resulting applications. System 20 includes one or more representative test subjects 22 at workstations 30. It should be appreciated that test subjects 22 and workstations 30 may be numerous or just singular with each subject/workstation participating in operations to be described hereinafter; however, only one set is shown to preserve clarity. Workstation 30 includes computing equipment 31 in a standard configuration capable of executing various software with corresponding memory and processing hardware. Equipment 31 includes Input/Output (I/O) 32, such as operator Input (UP) in the form of a standard computer keyboard 34 input device and mouse pointing device 36. Other UP 32 includes voice/sound command/monitoring subsystem 35, subject monitoring camera 37a (of a still image and/or video/moving image type), and subject sensor(s) 39—just to name a few. Sensor(s) 39 may be configured to monitor subject 22's body temperature, blood pressure, blood-oxygen content, other blood constituent(s), pulse, heartbeat, brain activity, muscle activity, or the like. I/O 32 may also include one or more forms of removable memory device, backup systems, etc. . . . . I/O 32 also includes operator Output (O/P) in the form of a graphical display device 38 with or without touchscreen input capability, an aural/audio loudspeaker output, and/or an audio line-out subsystem 38a to which headphones or earbuds may be connected in a standard manner—just to name a couple. It should be appreciated that the configuration of equipment 31 may lack at least some of the I/O components and/or may include others in different configurations. Collectively, in one example, workstations 30 are each used by a corresponding subject 22 to take a test and enter answers using various I/O 32 subsystems of workstation 30 as deemed appropriate for the particular testing exercise.

Equipment 31 of workstation 30 is also electronically in two-way communication with a computer I/O network that may be of a shared local type, a dedicated type, the internet, or a combination of these—and may be hardwired (electronically or optically for example), wireless, or a combination of these. System 20 also includes data mass media and server subsystem 50 also operatively coupled to network 40. In addition computing subsystem 60 is electronically coupled to network 40 in two-way communication therewith to analyze the performance of multiple subjects 22 suitable for dedicated monitoring, analysis, training, experimentation, data gathering, and oversight of data collected in test data server subsystem 50. Subsystem 60 may include one or more computer workstations each with some or all of I/O 32 described in connection with equipment 31. Software or hardware of subsystem 60 defines a number of timers 70 utilized to establish various timing benchmarks as more fully explained hereinafter.

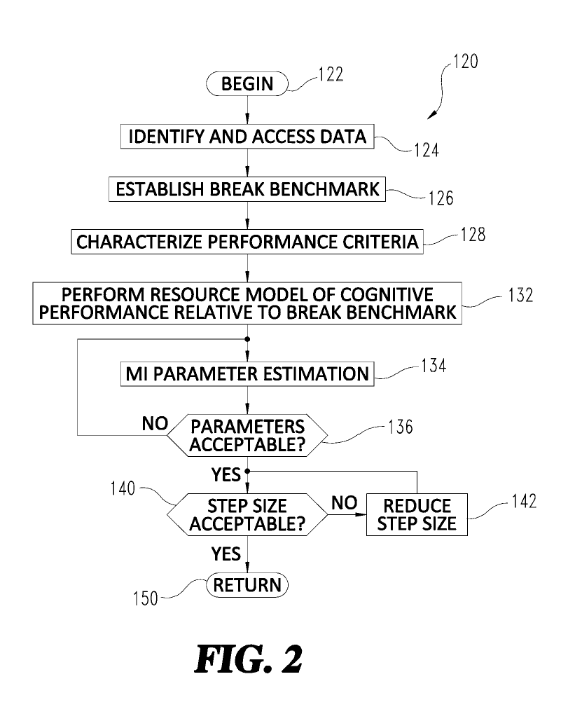

One of the significant challenges in evaluating cognitive performance of a large group of test subjects is the intrinsic heterogeneity of the group in terms of capability, educational background, test taking skills, speed/familiarity with which the operator input equipment can be used and manipulated, and the like. At least in part, these shortcomings were addressed by analyzing large-scale cognitive performance data as accumulated from a broad sample of test subjects taking common practice tests online. Referring additionally to the flow chart of FIG. 2, data evaluation procedure 120 is described in flow chart form; where like reference numerals refer to like features previously described. Procedure 120 begins at start flag 122 with identification of a large-scale cognitive performance data set from data server 50 in operation 124 that due to its nature, size, and breath is more amendable to the elimination of misleading outliers and reduction to a more meaningful data set for analysis.

Specifically, in operation 124 online practice standardized tests, the SAT (sometimes known as the Scholastic Aptitude Test), the ACT (sometimes known as American College Testing), and GED (General Education Development) were collected from a large cohort under real-world conditions. The data, was obtained from sponsorship by GROCKIT THE SOCIAL LEARNING COMPANY (which is accessible online at the worldwideweb site at grockit.com). GROCKIT is an online test prep service for students seeking to get their best potential score on the GMAT, SAT, ACT, GRE, and other tests required for college admissions. GROCKIT supplied data online to KAGGLE THE HOME OF DATA SCIENCE for the SAT, ACT, and GED tests (which may also be accessed online via the worldwideweb at kaggle.com/c/WhatDoYouKnow), which was prepared by KAGGLE to invite participants to perform an online competition. The SAT/ACT/GED data contains 2.8 million attempts by 180,000 users to answer 6000 questions. The data include the time a user started and stopped each question, the question's subject matter and outcome (i.e., whether the answer was correct). By focusing on the temporal aspect of performance, accuracy and learning decline as the test progresses can be observed. The results can be readily demonstrated and can be normalized to remove some of the variation between different test users as shall be further described hereinafter. It should be appreciated, the data of a given test user (subject 22) may be entered with workstation 30. The data input from all users/subjects 22 can be collected on data server 50 for selective access/analysis by subsystem 60 of system 20.

The full data are available at the KAGGLE site, along with the schema. The present investigation utilized the following entries: (a) outcome, (b) user_id, (c) question_id, (d) track_name, (e) round_started_at, and (f) deactivated_at. This information can determine when (round_started_at) a user (user_id) started each question (question_id) and when they answered it (deactivated_at) and if they answered correctly, incorrectly, skipped it, or abandoned it (outcome). For this analysis, attention was restricted to users who answered at least 15 questions, leaving about 180 thousand users that answered 6 thousand different questions, for a total of 2.8 million different attempts. By comparing correct answers with question_id, it can be determined that each question_id has a unique answer. If a question was a typical math or verbal question, a track name was assigned in either the math or verbal category as follows. Tracks ‘ACT Math’, ‘ACT Science’, ‘GED Quantitative’, and ‘SAT Math’ were tagged as math, while the tracks ‘ACT English’, ‘ACT Reading’, ‘SAT Reading’, and ‘SAT Writing’ were tagged as verbal. Unless indicated otherwise, separate statistics, rates, and outcomes were calculated for each user for the math and verbal categories, based on the observation that people often have different competencies in these areas. The time each subject 22 spent on the ith question, Tᵢ, was captured in expression (1) as follows:

Tᵢ=min(deactived_atᵢ,round_started_atᵢ+1)−round_started_atᵢ (1)

Because of idiosyncrasies of the grockit.com data, sometimes the data indicated that a user answered a question after they started the next question. In this case, consider the time when the user starts question i+1 as the definitive end of their work on question i. When a user abandons a question, e.g. shutting down their computer, stopping work entirely, or timing out on the question, the question is marked in the data as “'abandoned.” Consider the user to be working up to the point at which the data states the user deactivated the question, even if they abandoned the question. No attempt was made to guess whether the user was actually thinking about the question, nor did we attempt to place any artificial upper bounds on the time a user would spend on a question, to avoid investigator created bias. If a question was marked as skipped, this situation indicates that the user made the affirmative choice to skip a question. Although it is not technically a wrong answer, it is not the correct answer, and it is counted as such unless noted otherwise.

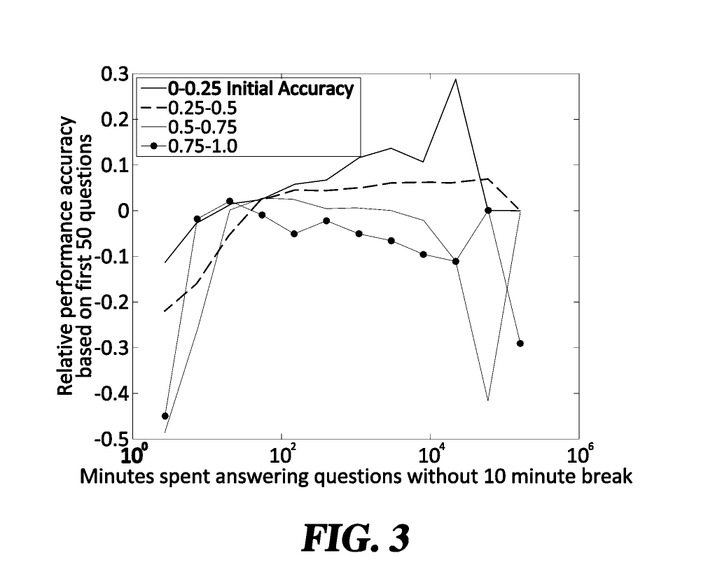

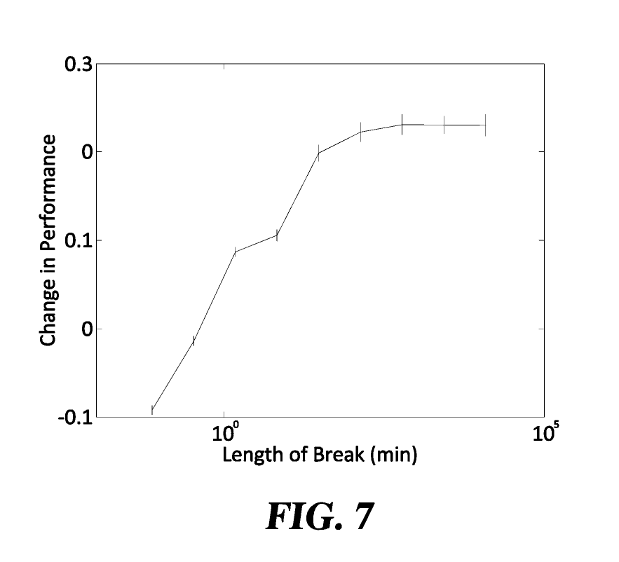

One of the main challenges in analyzing human performance is that individuals are extremely heterogeneous. It is desirable to account for this heterogeneity to remove excessive bias and variance from analysis. Properly characterizing variation between people requires sample sizes far larger than those typically available in a laboratory setting, making real-world online data about human behavior a useful tool for studying cognitive performance. Without such data, important trends are often obscured. For example, to test for the effects of cognitive depletion, one could track user performance on test questions over time, but this reveals no discernable trends as illustrated in the comparative graph of FIG. 3. Because the grockit.com data was produced under uncontrolled conditions, it is possible all users potentially begin working on practice tests under different initial circumstances—well-rested, well-fed, or otherwise. To reduce such variability, it is more plausible to identify a common temporal benchmark. Accordingly, the initial benchmark is taken from the first break of five (5) minutes—that is a period during which participants take a break from question answering for a period of at least five (5) minutes. This break may be for a number of different reasons such as fatigue, boredom, metal block or the like. Alignment relative to a break has been found to result in more uniform behavior instead of aligning users based on the time they started testing (it should be noted that it has been observed that a break time of longer than 5 minutes has not been observed to result in any marked difference) as depicted in the graph of FIG. 7.

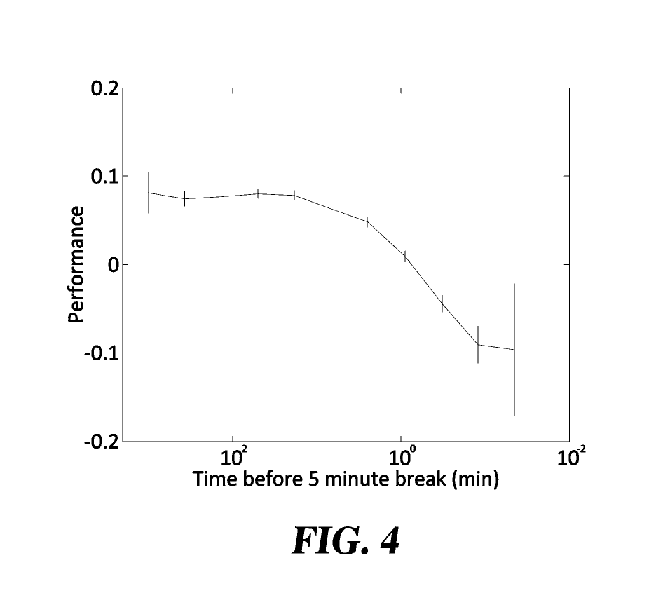

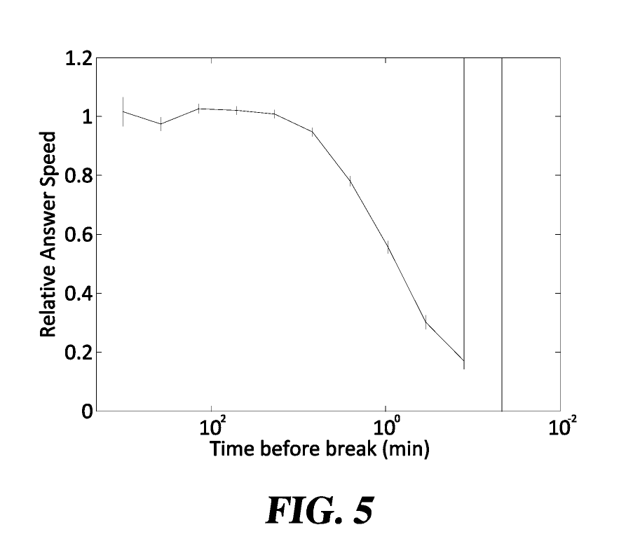

Further, to partially account for individual heterogeneity, first the probability is calculated that a user with net accuracy u correctly answers a question with net percentage (%) correct answers q, where the probability is represented by the expression P(u, q). Notice that when a question is difficult, q has a low value, meaning that few users have answered it correctly. Notably, after aligning users based on break time, a discernable link between performance and the time remaining before a break results, as illustrated in the graph of FIG. 4—even after controlling for user ability and question difficulty as further described hereinafter. It should be appreciated that even if the test subjects are not aware when they will take a break, the last question before the break is often never completed. To partially account for individual heterogeneity, the probability is determined that a user with net accuracy u correctly answers a question with net percentage (%) correct answers q, represented as “performance” [symbol] (u, q). Notice that when a question is difficult, q has a relatively low value, meaning that few users will likely have answered it correctly. User's performance [symbol] on an attempt “a” is represented by [symbol] =δₐ− [symbol] (u, q), where δa=1 if the user answered the question correctly, and zero (0) otherwise. In this way, the outcomes can be compared of different users answering different questions. An average performance of < [symbol] >=0 indicates a user answers questions correctly at the same rate as we expect for those user/question combinations. Negative performance [symbol] <0 signifies a user is under-performing, and positive performance [symbol] >0 signifies a user answering questions correctly more frequently than expected (outperforming). As users approach a break, their performance decreases as illustrated in FIG. 4 and the speed at which they answer questions also decreases until very near the break time as illustrated in FIG. 5. Accordingly, a method of evaluating the relative performance difficulty of specific questions and an overall range of positive (>0), average (0), and below average (<0) performance [symbol] parameters can be characterized.

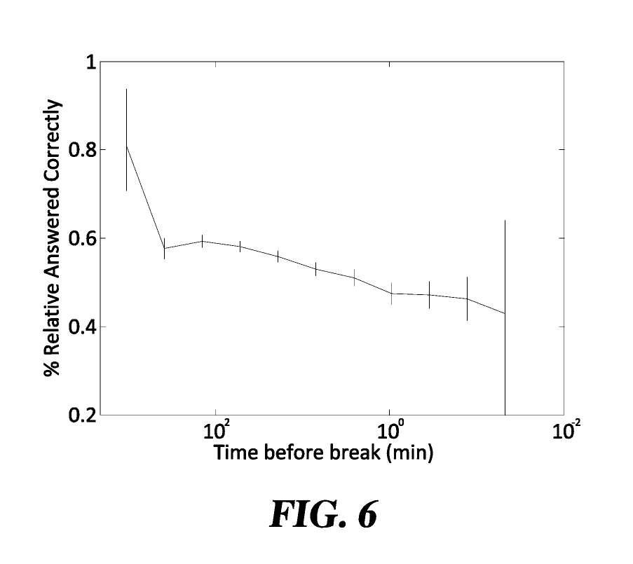

After answering a question on GROCKIT, the user/test subject is presented with the correct answer, regardless of the outcome, and thus he or she has the opportunity to learn both the solution method and the corresponding answer. If the user is presented the same question again, it will have the same answer. To estimate learning, the next question is identified with the same question-id attempted by the user. The calculation is limited only to questions for which the user actually provided a response, such that the user had the opportunity to be exposed to the correct answer. If the users learn (or at least remember) the questions from previous attempts, they should be able to answer them correctly upon repeat exposure. However, a systematic decline in users' accuracy as a function of time before a break was observed as illustrated in FIG. 6. Conversely, the length of time to a break is highly correlated with performance.

As the time from the end of one question to the start of the next question increases, the user's relative performance increases as illustrated in FIG. 7. That is, while the absolute performance does not vary significantly as a function of time between questions (or may even decrease due to “warm-up” requirements described hereinafter), the relative change in performance from the previous question to the next one increases with the between-question time interval. This result suggests that longer breaks between questions are associated with under-performance, and the worse the under-performance, the longer the break the user takes, recovering the performance after the break. The same trend holds for learning: users appear to take longer breaks after their ability to learn the answer has decreased (not shown).

As mentioned earlier, a significant technical challenge in modeling human data is the large variation among users. In one sample, users differ substantially, among other things, in: (a) the number of questions they attempt, (b) the length of time they worked without a break and (c) the speed of answering a question. To handle individual heterogeneity, each test user was characterized by two performance-independent parameters. First, to quantify whether a test user was faster or slower than average, the mean time taken by each user was measured to answer a question correctly relative to the population's average time to answer that question correctly. Second, as a proxy for the maximal amount of the available cognitive resources, the longest time each test user spent answering a question correctly was determined. The rates of each user were scaled based on these two numbers. This user-specific scaling is based only on the observed time series of user's answers and is not an explicit fitting step nor post-hoc constraint on user performance (beyond knowing they answered at least one question correctly). This scaling procedure resulted in significant improvement of Mutual Information (MI) estimates (>5×improvement) and demonstrates that incorporation of underlying user heterogeneity is readily justified to provide a meaningful study of cognitive depletion. The role of MI is described in greater detail hereafter.

To account for user heterogeneity, it was decided to fit one set of kinetic parameters, but each parameter was scaled for each user based on performance-independent observable user behavior. First, for each user, the longest time it took to answer a question correctly was measured as indicated in expression (2) as follows:

$${T}_{L} = {max}_{i ∈ correct} {T}_{i} \qquad ( 2 )$$

where: Tᵢ is the time the user spends answering question i, as determined from expression (1). This approach only assumes that the user answered at least one question correctly. Although the user may have answered the question correctly purely by chance, it is hypothesized that the user shall only be able to answer questions correctly when they have sufficient cognitive reserves and that the longest time they spend on a question that was answered correctly scales with the total depth of their cognitive resources. Second, for each user the average time was measured that it took to answer a question correctly, relative to the average time it took all users to answer that question correctly. More specifically, Tᵣ reflects a user's speed relative to average when attempting to answer a question as set forth in expression (3) as follows:

$${T}_{r} = \frac{1}{{N}_{c}} {Σ}_{i ∈ correct} \frac{{T}_{i}}{〈 {T}_{i} 〉} \qquad ( 3 )$$

where: N_{c} is the number of questions the user answered correctly, Tᵢ is the time the user spent on the ith question, and <Tᵢ> is the mean time all users took to answer that same question correctly. Thus, Tᵣ reflects if a user is faster than average or slower than average when attempting to answer a question. Correctly answered questions generally remove any other questions subject to skipping, guessing (most guesses will be incorrect), and abandoning. User parameters were constrained to fall between the 5th and 95th percentiles, which were 33 s and 200 s respectively for the math T_{L}, 29 s and 240 s for verbal the T_{L}, 0.46 and 1.6 for the math Tᵣ, and 0.45 and 1.7 for the verbal T. Parameters falling below this range were set to the 5th percentile for that value, while those falling above the range were set to the 95th percentile. This procedure removes pathological effects due to users who only guess, users who answer questions abnormally slow, and the like. For the model fitting procedure (detailed hereinafter), all of the users rates were scaled as k→k/Tᵣ, where: Tᵣ is specific to each user. In addition, further scaling included:

$${B}_{max} \to {B}_{max} \frac{{f}_{0}}{{T}_{r}} ,$$

where expressions (4) and (5) are set forth as follows:

$$\begin{matrix}if ρ = 1 \text{:} log ( {T}_{r} + 1 ) & ( 4 ) \\ if ρ = otherwise \text{:} \frac{( {( {T}_{L} + 1 )}^{ρ} - ( {T}_{L} + 1 ) )}{{( {T}_{L} + 1 )}^{ρ} ( ρ - 1 )} & ( 5 )\end{matrix}$$

In this way, rates for faster users who were able to work longer successfully were different from slow users or users who could not maintain high performance levels over extended periods.

This investigation has led to the discovery that various performance criteria depend on the time until the next break, and further that cognitive performance is inversely related to the performance time and the approach of a break to “recharge” for further test activity. It has also been found that this relationship may result from limited cognitive resources—namely a limited-resource model in support of brain-intensive activity of the type occurring with concentrated test-taking. This resource-limited theory is suggestive of neuroenergetic modeling. To understand these observations and predict performance, two potentially related neuroenergetic kinetic models of cognitive resource depletion have been found applicable. Each question/answer pair has a unique id, so questions with the same id will have the same answer. A single resource model, was discovered from consideration of certain work exploring the link between glucose and finite willpower in preexisting literature. The model considers a single resource P, that decreases while the user is working (i.e., attempting to answer a question) and recovers during periods of rest (i.e., time interval between question-answer attempts). It is proposed that resource P is the primary driver of performance. The neuroenergetic glucose mechanism, while perhaps not completely explained or otherwise established, is one of the better understood models for supplying the brain with energy during intense cognitive events. The general form for the one-resource P model may be given by expression (6) as follows:

\dot{A}(t)=−ω(A,t)δ(t)+r₁(A,t)(1−(t)) (6)

where: ω₁ is a function representing kinetics of depletion during work, r₁ is the kinetics of recovery, and δ(t)=1 when the user is recorded as working on a question and zero (0) otherwise. It should be understood that either the first term or the second term of the sum is zero (0) at any given time, leaving the other term to govern the result of expression (6). Accordingly, only the kinetics of depletion (first term) or the kinetics of recovery (second term) applies. The precise form of the kinetic functions were chosen to represent enzyme-catalyzed (Michaelis-Menton) kinetics under heterogeneous conditions commonly found in the literature. That is, one expects brain activation to be broadly distributed and inhomogeneous, and this result is represented in the functionals chosen for the model. Specifically, the following expressions (7) & (8) indicate rates:

$$\begin{matrix}- {ω}_{1} ( A , t ) = \frac{k}{{t}^{ρ}} \frac{A ( t )}{( {k}_{m} + A ( t ) )} & ( 7 ) \\ {r}_{1} ( A , t ) = \frac{{k}_{r}}{{t}^{ρ}} ( {A}_{max} - A ( t ) ) & ( 8 )\end{matrix}$$

which depend on a number of constants including kₘ, k, kᵣ, and ρ allows for enzyme-mediated reaction with anomalous diffusion.

Emerging evidence suggests that glucose may not be the only energy source for neurons engaged in intensive activity. Instead, an additional source may be at work. Indeed, one candidate is the lactate shuttle metabolism. This mechanism may play a prominent role. It has been observed in connection with this investigation that the lactate mechanism dovetails well as a secondary resource S (also represented by “B” in various expressions) with glucose as the primary resource P (A). It undergoes depletion with significant brain activity from standardized testing. Whether a single resource or two resource model, routine 120 of FIG. 2 captures such modeling in operation 132.

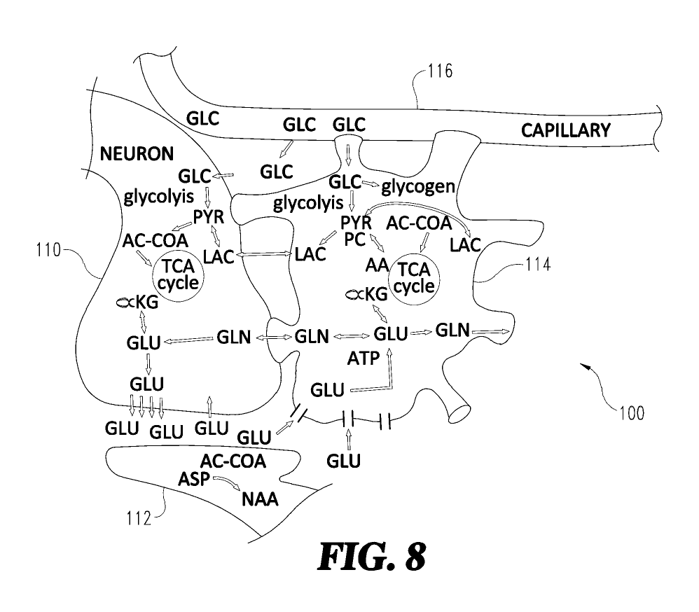

Turning additionally to FIG. 8, a partially diagrammatic representation of central nervous system cells 100 of the type that might play a role in cognitive performance are illustrated with abbreviated reactions corresponding to the primary glucose and secondary lactate neuroenergetic models P and S, respectively; where like reference numeral refer to like features previously described. FIG. 8 more specifically illustrates presynaptic neuron 110, postsynaptic neuron 112, astrocyte glial cell 114 and capillary 116. Capillary 116 serves as the ultimate source of nutrients and as a pathway to remove waste products in relation to the energy generation from either a primary P or secondary S source. Further reference should be made to the abbreviations, acronyms, and definitions set forth in the Background for terminology used in the reactions set forth in FIG. 8.

Correspondingly, a two-source model based on these primary P and secondary S cognitive depletion models are set forth as follows. This modeling considers a primary resource P (also represented by “A” in the expressions), which is responsible for performance but is normally low during rest conditions. Engaging in the task consumes resource A (P), but also causes conversion of a secondary resource B (S) into A as reflected in expressions (9) & (10):

\dot{A}(t)=−f(A,t)δ(t)+ω₁(A,B,t)δ(t) (9)

\dot{B}(t)=−ω₂(A,B,t)δ(t)+r₂(B,t)(1−δ(t)) (10)

where secondary resource S is also represented by “B” in expression (9)-(13), f is the rate of consumption of primary resource A, ω₂ is the conversion rate of a secondary resource S into a primary resource P, and r₂ is the rate of recovery for secondary resource S. The functions of the two-resource model are reflected in expressions (11)-(13):

$$\begin{matrix}f ( A , t ) = \frac{{k}_{ω}}{{t}^{ρ}} \frac{A ( t )}{( {K}_{A} + A ( t ) )} & ( 11 ) \\ {r}_{2} ( B , t ) = \frac{{k}_{r}}{{t}^{ρ}} ( {B}_{max} - B ( t ) ) ( 1 - δ ( t ) ) & ( 12 ) \\ {ω}_{2} = ( A , B , t ) ≡ \frac{{k}_{b}}{{t}^{ρ}} \frac{( 1 - A ( t ) ) B ( t )}{{K}_{B} + B ( t )} & ( 13 )\end{matrix}$$

The functional form of the parameters, although nontrivial, is a natural extension of the one-resource model, allowing for more complex and anomalous enzyme kinetics.

Both models are parameterized by rate constants, which were estimated by maximizing the explanatory power of the model, i.e., by maximizing the Mutual Information (MI) between the dynamic values of A or A, B (alternatively designated primary and secondary resources P and S, respectively) and the outcome of the corresponding question (correct or incorrect). These rate constants include: k, kᵣ, ρ, kₘ, kᵣ, kω, k_{b}, kᵣ, and Bₘₐₓ. In addition, k_{A} and k_{B} were assigned values. Mutual information MI (formerly sometimes known as transinformation) of two random variable is a measure of the variables' mutual dependence—being a reduction in entropy of a random variable achieved by knowing another variable. MI determines how similar the joint distribution of the random variables X and Y (p(X,Y)) is to the products of factored marginal distribution p(X)p(Y). It is a more generalized measure of correlation coefficient; where correlation coefficient is limited to real-valued random variables. The application of mutual information MI does not require knowledge of the precise way that hypothetical resources translate into performance to find parameters that maximize the explanatory power of the model. The model fitting procedure is described as follows:

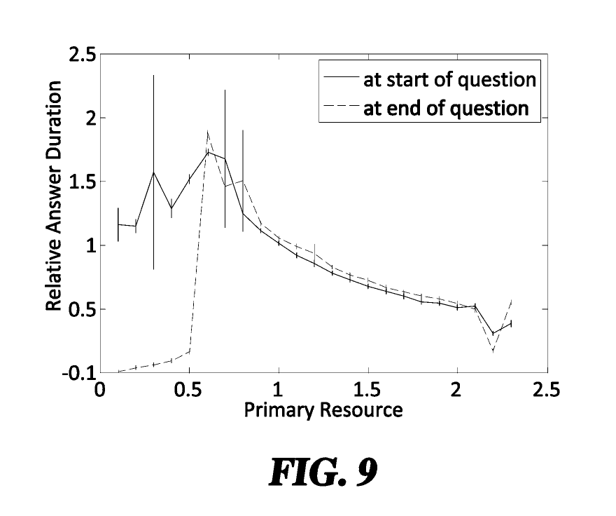

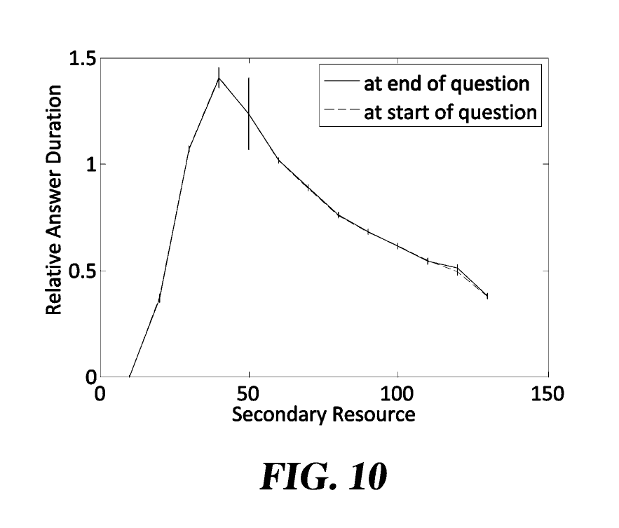

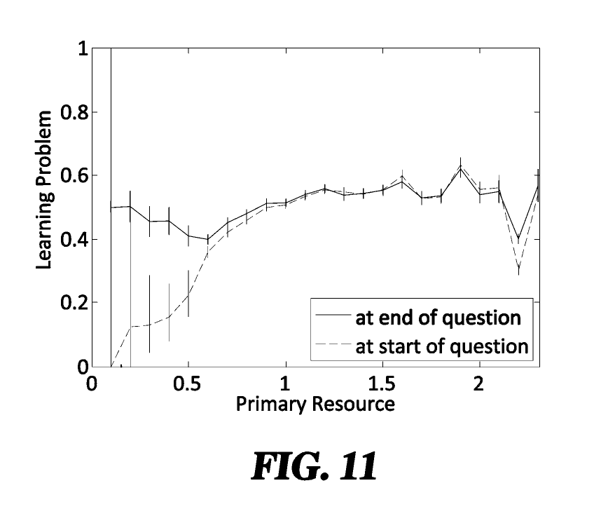

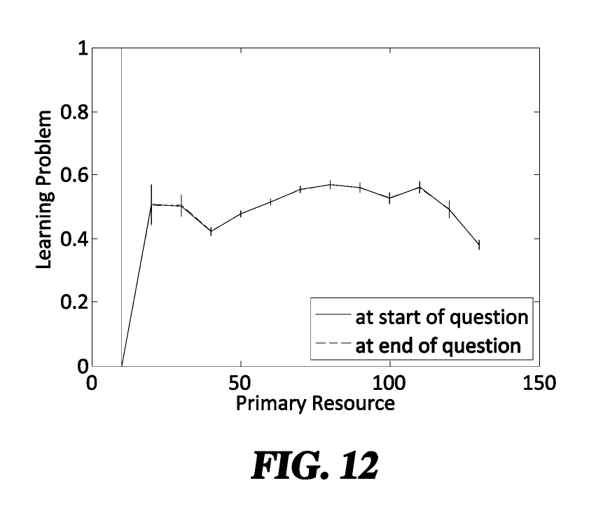

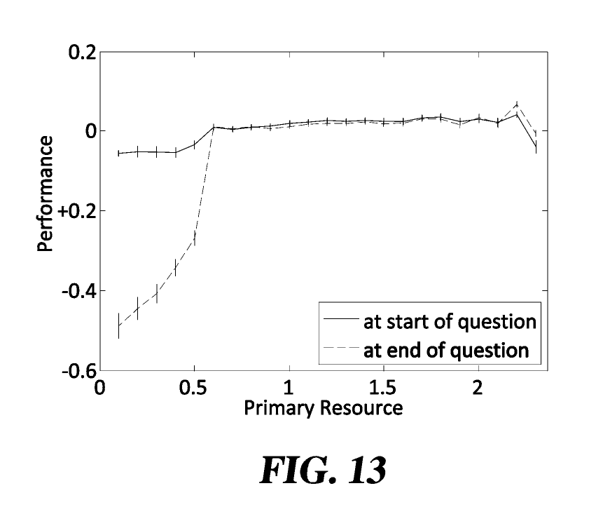

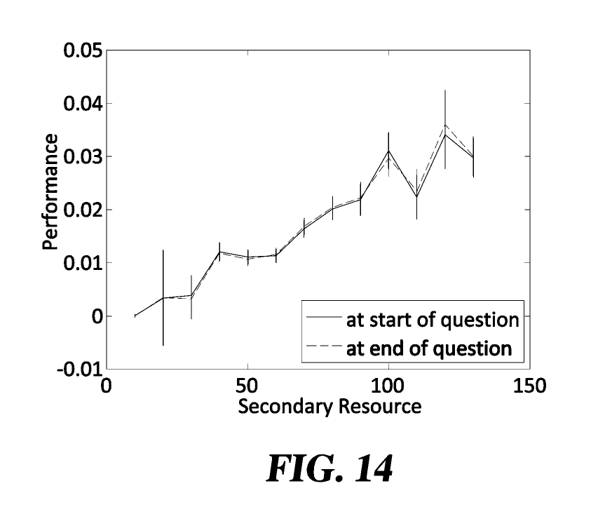

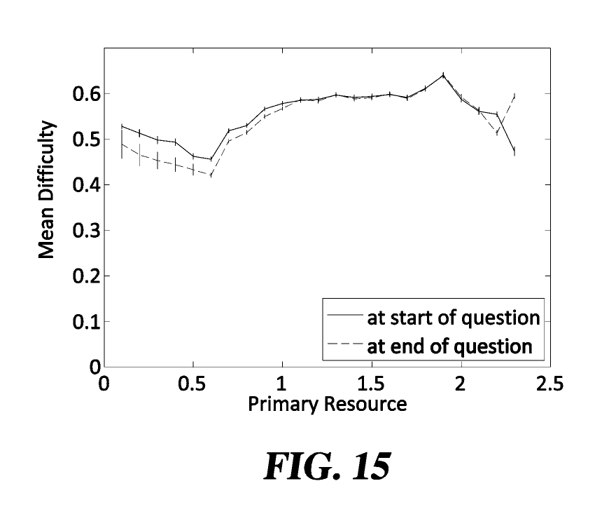

After applying the models, they were used to estimate the levels of resources P and S in users at the beginning and end of question-answer attempts. FIGS. 9-15 shows how various performance characteristics depend on the primary P and secondary S resources of the two-resource model. Answer-speed is determined with both primary and secondary resource models as illustrated in FIG. 9 and FIG. 10, respectively. FIG. 9 is a graph of relative answer duration (normalized value on vertical axis) versus relative primary resource P usage (horizontal axis). FIG. 10 is a graph of relative answer duration (normalized value on vertical axis) versus relative secondary resource S usage (horizontal axis). FIG. 11 is a graph of learning probability (vertical axis) versus relative primary resource P usage (horizontal axis). FIG. 12 is a graph of learning probability (vertical axis) versus relative secondary resource S usage (horizontal axis). FIG. 13 is a graph of performance (vertical axis) versus relative primary resource P usage (horizontal axis). FIG. 14 is a graph of performance (vertical axis) versus relative secondary resource S usage (horizontal axis). FIG. 15 is a graph of mean difficulty (vertical axis) versus relative primary resource P usage (horizontal axis). The more resources test users possess, the faster they answer questions. As resources decline, answer speeds decline, but below a certain threshold users answer questions very rapidly relative to the average time they spend on the questions. At these levels of the primary resource, relative accuracy also declines rapidly. Together, these observations suggest that when primary resources are too low, users guess answers—answering quickly, but at the expense of accuracy as resources have declined. Similarly, when their primarily resources are low at the end of the question (when the correct answer is revealed), users may have more difficulty remembering or learning correct answers to questions, as shown in FIG. 11. Note that performance does not appear to suffer as much when the primary resource P is low at the beginning of the question, which could indicate either depletion or the user starting off in a cold state, i.e., with low initial resources. In the former case, performance appears negatively impacted by depleted resources, while in the latter case, primary resource P levels appear to increase over the course of the question-answer attempt due to conversion of the secondary resource S, which will result in improved performance after a brief “warm-up” period. The two-resource model, where the driver of cognitive performance is drawn from a secondary resource S, provides more congruent performance-related metrics relative to available resources P and S than the one-resource model. TABLE I reports Mutual Information MI between actual user performance and resources estimated by the one-resource and two-resource models as follows:

TABLE I

| | Two-Resource Model | | | One-Resource Model | | |
|---|---|---|---|---|---|---|
| Quantity | Bits | Std. Dev. | % Entropy | Bits | Std. Dev. | % Entropy |
| MI(A; R) | 0.12 | 0.015 | 12% | 0.04 | 0.006 | 4% |
| MI(L; R) | 0.10 | 0.02 | 16% | 0.03 | 0.014 | 3% |
| MI(T; R_{b}) | 0.51 | 0.05 | 8% | 0.15 | 0.01 | 2% |
| MI(ΔT; R) | 1.18 | 0.06 | 16% | 0.27 | 0.016 | 4% |

where: For TABLE I, abbreviations used are MI (Mutual Information), A (answered correctly), R (resources at beginning and end of question), L (learned correctly), AT (time until next question), T (time spent on question), and Rb (resources at beginning of question). Mutual information MI is the reduction in entropy (uncertainty) of random variable X achieved by knowing another variable Y. For example, how much information about an individual's performance (X) is obtained by knowing the amount of cognitive resources available to him or her (Y)? MI is defined as MI(X,Y)=H(X)−H(X|Y), where H(X) is the entropy of the random variable X, and H(X|Y) is the entropy of X given Y, and it is typically measured in bits. Mutual information MI has the property that MI(X, Y)=MI(f(X), g(Y)) for invertible functions f and g, so it is not necessary to know the precise way that hypothetical resources translate into performance to find parameters that maximize the explanatory power of the proposed models. In contrast, optimizing a regression model or Pearson correlation for parameter estimation requires not only correctly modeling resources but also knowing how those resources quantitatively translate into predicted performance, because R² is only maximized when predicted and observed results have an affine relation. Contrast this to MI, where a large MI implies large explanatory power, even if the precise mapping from X to Y is not known. To calculate mutual information MI, we utilized a variation of the method described in: D. P'al, B. P'oczos, C. Szepesv'ari; “Estimation of R′enyi Entropy and Mutual Information Based on Generalized Nearest-Neighbor Graphs” arXiv: 1003.1954v2 [stat.ML] 25 Oct. (2010), which is hereby incorporated by reference in its entirety herein.

As shown in FIG. 2, routine 120 proceeds from operation 132 to model estimation operation 134. More specifically, to calculate MI(X,Y), a copula transform was first performed on each dimension of X and Y, and then the quantity indicated by expression (14) was calculated:

MI(X,Y)=−H([XY])+1/10Σᵢ₌₁¹⁰H([X,Yᵢ^{˜}])+H([Xᵢ^{˜},Y])−H([Xᵢ^{˜},Yᵢ^{˜}]) (14)

where: H([XY]) is the R′enyi entropy with R′enyi-exponent taken as 0.99999 (that is, a very close approximation to Shannon entropy), calculated according to P′al et al. (previously incorporated by reference). Xᵢ^{˜} is a shuffled version of X, such that any correlation between dimensions of X or Y are destroyed, and i denotes the ith shuffle. This version of mutual information (MI) was chosen because it demonstrated the most robustness. Normal calculations of mutual information MI, calculated directly as H(X)+H(Y)−H([XY]) subtract entropy calculated from low-dimensionality spaces from entropy calculated from high-dimensional spaces. Because bias in entropy calculations vary with dimension, the shuffled version helps to cancel out systematic dimensional biases.

From operation 134, routine 120 of FIG. 2 advances to conditional 136, which tests whether the model fitting estimation/determination meets acceptable requirements. Before engaging routine 120 further, one nonlimiting example of model fitting is explained. To determine the parameters to use for estimating resource levels, the data was first divided into training and test sets. The training set comprised 250 users, each making at least 500 attempts at one or more questions. Out of the training set, the only measurements were for the performance between the 2nd attempt and the 5000th attempt (should the user make more than 5000 attempts), to avoid overweighting the statistics with attempts from a small handful of users. The test set comprised users who made at least 25 attempts, answered at least 20% of the question correctly, and were not included in the training set. The training set was used to find parameters, and the results in TABLES I and II and FIGS. 9-15 were all produced from the test set.

To find parameters, for each user in the training set, the resources were evaluated at the beginning and end of each attempt. For the one-resource model, the following quantity was calculated according to the expressions (15-17):

$$\begin{matrix}\dot{A} ( t ) = - {ω}_{1} ( A , t ) δ ( t ) + {r}_{1} ( A , t ) ( 1 - δ ( t ) ) , & ( 15 ) \\ {ω}_{1} ( A , t ) = \frac{k}{{t}^{ρ}} \frac{A ( t )}{( {k}_{m} + A ( t ) )} , & ( 16 ) \\ {r}_{1} ( A , t ) = \frac{{k}_{r}}{{t}^{ρ}} ( 1 - A ( t ) ) ; & ( 17 )\end{matrix}$$

which depend on a number of constants including kₘ, k, kᵣ, and ρ allowing for enzyme-mediated reaction with anomalous diffusion.

Correspondingly, a two-source model based on these primary P and secondary S cognitive depletion models are set forth as follows. This modeling considers a primary resource P (also represented by “A” in expressions), which is responsible for performance but is normally low during rest conditions. Engaging in the task consumes resource A (P), but also causes conversion of a secondary resource B (S) as reflected in expressions (18)-(20):

$$\begin{matrix}\dot{A} ( t ) = θ [ A , B ] - \frac{{k}_{ω}}{{t}^{ρ}} \frac{A ( t )}{( {K}_{A} + A ( t ) )} & ( 18 ) \\ \dot{B} ( t ) = - θ [ A , B ] + \frac{{k}_{r}}{{t}^{ρ}} ( {B}_{max} - B ( t ) ) & ( 19 ) \\ θ [ A , B ] = δ ( t ) \frac{{k}_{b}}{{t}^{ρ}} \frac{( 1 - A ( t ) ) B ( t )}{{K}_{B} + B ( t )} ( 1 - δ ( t ) ) & ( 20 )\end{matrix}$$

As mentioned above, resources for math and verbal questions were calculated separately, so two different independent sets of resources were calculated for each user. When a user is working on a math question, it is considered resting for the verbal resources, and vice versa. For the purpose of comparing resources to performance, comparisons were only made for the resource that matches the type of question. So, if the user is attempting to answer a math question, the outcome is compared with the resources connected to math. The parameters were estimated/determined within acceptable limits using the NLopt optimization library with Constrained Optimization By Linear Approximation (COBYLA), which is a numerical optimization method for constrained problems where the derivative of the objective function is not known.

That is, COBYLA can find the vector \vec{x}εS with S⊂Rₙ that has the minimal (or maximal) f\vec{(x)} without knowing the gradient of f. In general, this algorithm works by iteratively approximating the actual constrained optimization problem with linear programming problem solving techniques. During an iteration, an approximating linear programming problem is solved to obtain an estimation candidate. The candidate estimate evaluated using the original objective and constraint functions yields a new data point in the optimization space as reflected in the test of conditional 136 of routine 120. If the test is negative (No), routine 120 of FIG. 2 loops back to operation 134 to perform another interation, returning to conditional 136 to re-perform the test of the parameter estimate. The estimated information is used to improve the approximating linear programming problem used for the next iteration of operation 134. This loop of routine 120 repeats, reiterating with new parameters until the estimate/solution meets expectations—such that the test of conditional 136 is affirmative (Yes). Next, routine 120 proceeds to operation 140 to reduce the step size and correspondingly refine the search. Next, routine advances to conditional 142 to test if the step size is acceptably small. If the test of conditional 142 is negative (No), routine 120 returns to operation 140 to iteratively decrease the step size until the test of conditional is affirmative (Yes), at which point routine proceeds to operation 150 to return from routine 120 until called again. The resulting parameters were all constrained to be between 0.0001 and 2.0. The results of the fitting were as follows: for the one factor model, k=0.078, kᵣ=0.21, ρ=1.0, Kₘ=0.44867; and for the two factor model k_{w}=0.003, k_{b}=0.118, kᵣ=0.00125, Bₘₐₓ=0.27, ρ=0.03. In addition, we fixed K_{A}=0.858 and K_{B}=0.1, which were values taken from M. Cloutier, F. B. Bolger, J. P. Lowry, P. Wellstead, “An integrative dynamic model of brain energy metabolism using in vivo neurochemical measurements,” Journal of Computational Neuroscience 27 (3), 391-414, which is hereby incorporated by reference as if set forth herein in its entirety.

The two-resource model accounts for approximately 12% of the variance in user's performance on a question, compared to the one-resource model, which just accounts for approximately 5% of the variance. Similarly, the two-resource model explains approximately 16% of uncertainty in whether or not a user will answer the same question correctly in the future (learning). In contrast, the one-resource model accounts for only about 3% of uncertainty in learning. The two-resource model also explains about 16% of uncertainty in how long a user will spend between questions and about 8% of how long a user will spend answering a particular question, knowing only the resources available at the beginning of the question, compared to the one-resource model, which explains about 4% and 2% of the variance, respectively.

To check whether the resource depletion model merely shadows a different, non-resource feature by coincidence, alternative hypotheses were tested. A commonly proposed explanation for performance decrease assumes that users are sensitive to their near-term success, for example, measured by the fraction of the previous five questions they answered correctly. Under this alternative to resource depletion modeling, when performing successfully, test subjects may be highly motivated and engaged, but when success declines they may become discouraged and inattentive. In other words, under this alternative, could the resource model simply be a proxy for the positive or negative feedback a user receives by answering questions correctly or incorrectly? This alternative hypothesis was tested with Conditional Mutual Information CMI, which is defined as CMI(X, Y|Z)=MI(X, [Y Z])−MI(X, Z). If random variable Y (e.g., current available resources) is merely a proxy for random variable Z, (e.g., near-term success), then CMI(X, Y|Z) will be smaller than MI(X,Y). In the extreme case where Y=Z, CMI(X, Y|Z)=0. If, on the other hand, Y and Z together explain X (e.g., performance) better than either alone, than CMI(X, Y|Z) will be larger than MI(X,Y). Accordingly, under this analysis, the two-resource model cannot be explained by near-term success: the CMI between performance and resources were conditioned on successfully answering the previous five questions is statistically unchanged from MI between performance and resources, as shown in the following TABLE II:

TABLE II

| Alternative Hypothesis | Bits | Std. Dev. |
|---|---|---|
| CMI(L; R\|ΔT) | 0.14 | 0.03 |
| CMI(L; R\|T) | 0.10 | 0.03 |
| CMI(A; R\|H) | 0.11 | 0.02 |
| CMI(A; R\|E) | 0.18 | 0.02 |
| CMI(A; R\|T) | 0.14 | 0.02 |
| CMI(A; R\|D) | 0.14 | 0.02 |
| CMI(ΔT; R\|H) | 1.36 | 0.06 |
| CMI(L; R\|ΔT) | 0.14 | 0.03 |

Where: abbreviations for TABLE II are CMI (Conditional Mutual Information), A (answered correctly), R (resources at beginning and end of question), L (learned correctly), ΔT (time until next question), T (time spent on question), H (average performance on previous 5 questions), E (parameters defining user), and D (difficulty of question).

The user's parameters that fit the model do not explain the mutual information MI between performance and resources, nor does the difficulty of the question, and nor the time spent on the question. It is next determined if learning can be explained by the time spent on a question or the time until the next question (i.e., potentially forgetting the answer). It can be concluded that the explanatory power of resources is not a simple proxy for any of the alternative explanations. In fact, there is a distinct synergy in knowing both of these features. For example, the intuition that a test subject may get discouraged by a streak of poor performance, leading them to take a longer break, may also be correct, but it does not rule out the resource model. Results of the two-resource model suggest that performance on practice standardized tests is tied to levels of cognitive resources. As these resources are depleted by sustained mental effort, performance and answer speeds decline, and users have more difficulty learning correct answers. The model also suggests that performance may be tied to a resource that is normally low when the user is not on-task, requiring a “warm-up” period to raise its level sufficiently. The two-resource model better explains observed performance on test questions than alternative theories. Even in uncontrolled environments of at-home practice tests, it succeeds at explaining over 10% of the uncertainty in user performance. While small in absolute size, this effect is significant to the user, because it changes what is normally roughly 50:50 odds of answering a question correctly into roughly 70:30 odds as further described hereinafter. Therefore, it may be possible to improve performance on standardized tests simply by better managing available estimated resources. The single resource glucose model cannot explain as many auxiliary observations nor produce a similar improvement in question answer odds as the two-source models. This approach may explain reported inconsistencies linking ego depletion to levels of glucose in laboratory studies. The findings suggest an alternate mechanism, where the driver for cognitive performance is drawn from a secondary resource S. This observation is consistent with how the lactate shuttle is believed to function in brain metabolism. In addition, motivational factors were specifically tested, which did not present significant explanatory power compared to the two-resource model. By applying data analysis and modeling techniques to large-scale practice test data, it has been discovered that cognitive depletion may account for a significant portion of the observed variance in test performance. By accounting for an individual's cognitive resources, ability improves to predict cognitive performance and devise strategies for improving it.

Odds adjustment to provide entropy for a binary outcome (correct or incorrect) is defined as S=−p log₂(p)−(1−p) log₂(1−p), where p is the probability of answering the question correctly. For all users, averaging over all questions, the total entropy for getting the question correct or not is roughly 1 bit, meaning without knowing anything about the user or the question, the user has a roughly 50:50 chance of getting the question correct. As previously reported, the two resource model accounts for roughly 12% of performance variance, so once resources at the beginning of the question are known, the remaining entropy is 0.88 bits. Thus, 0.88=−p*log₂(p*)−(1−p*) log₂(1−p*), where p* is the probability of answering the question correctly (or incorrectly). Thus, p* is 0.7 or 0.3. Therefore, knowing the resources, or estimating them using the model, changes the odds the user answers that question correctly 50:50 to 70:30 (high resources) or 30:70 (low resources), even without incorporating anything else about the question or user. The user may then make adjustments in their test-taking strategy to improve the overall score.

In another embodiment, computerized game data is evaluated to confirm various observations made in connection with the standardized test data. Among other things, salient game metrics include a “kill/death” ratio for each player. Further, the game data evaluation confirms efficacy of various aspects of routine 120 in relation to resource-based neuroenergetic modelling—like improved player performance after taking a gaming break. In one game model, it is represented by a bar that changes state as the game progresses. At the start of the game, the bar is “fully charged” with maximum resources available as represented by a solid color. The bar also includes divisions by dashed line into three generally equally sized regions corresponding to good, intermediate, and bad performance with respect to game resources. As the game progresses, the expenditure of resources is indicated by a corresponding reduction in a portion of the bar as indicated by a change in bar color and/or pattern. This reduction occurs from the full state towards the dashed line separating the good and intermediate regions. Critically low resources (in the “bad” region below the dashed line separating the bad and intermediate regions is further highlighted by a more striking color and/or pattern. The bar representation further utilizes embedded arrow heads in numbers that increase with the degree/rate of resource increase or decrease. The game modeling also distinguishes between short-term and long-term trends, which both appear to recover after a break, too. In addition, the game model provides kill/death rate predictions (changes).

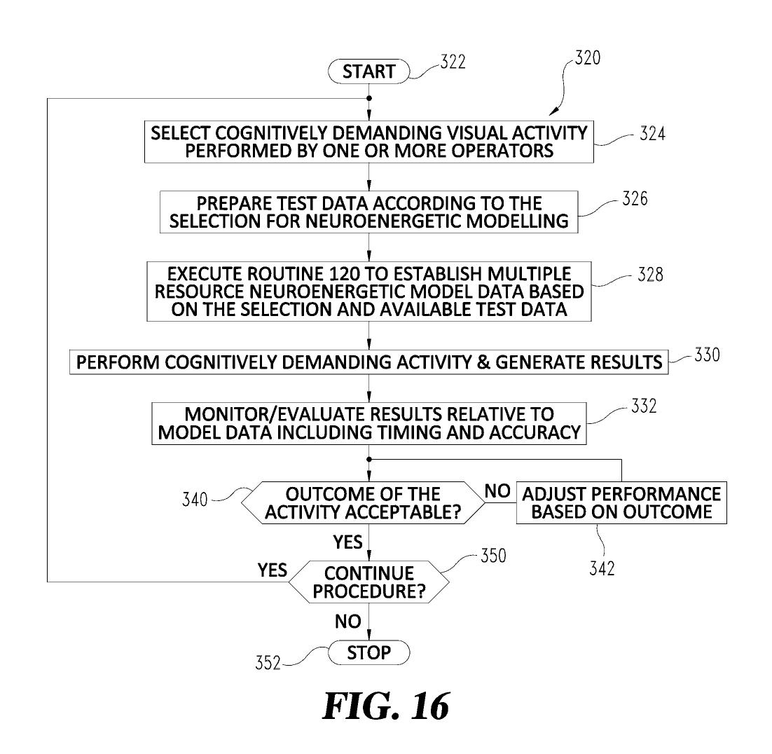

Referring additionally to FIG. 16, a flow chart of application procedure 320 is depicted that includes some aspects previously described and introduces others; where like reference numerals refer to like features previously described. Procedure 320 begins with start stage 322. From stage 322, procedure 320 advances to operation 324. In operation 324, selection is made of a suitable platform for analysis in accordance with the present application. Like the platforms associated with standardized test data and computerized gaming data, among those platforms particularly suitable to monitoring, evaluating, applying, enhancing, and/or analyzing in accordance with the principles of the present application are those that are cognitively demanding or cognitively intensive to an operator. Typically, such platforms require the operator to understand and/or interpret changing visual information (textual and/or graphic) often presented with some type of electronic display and take corresponding action—usually by providing corresponding operator input to equipment 31 associated with the display. In some instances, operators are evaluated relative to associated minimum standards for a particular platform type and/or setting.

With this background in mind, some platforms suitable to such applications are civil and military air traffic control systems that typically include at least one display and associated operator input devices. Such displays may incorporate input from one or more forms of aerial, airborne, or ground radar devices, transponders, transceivers, Global Positioning Satellite (GPS) devices that transmit location or the like; and often include a suite of corresponding operator input devices. Along similar lines are military threat/target displays and associated operator inputs. Still other platforms may include satellite imagery displays, and/or medical imagery devices (such as X-ray machines, Magnetic Resonance Imaging (MRI) devices, Computer-aided Tomography (CT) scanners, ultrasound machines, and/or PET scanners—just to name a few); each usually accompanied by suitable operator input devices. Another imaging system particularly suitable to treatment under procedure 320 are security scanning devices that are capable of presenting an image of the interior (to a varying extent) of a traveler's carry-on belongings by X-ray, millimeter wavelength, or otherwise—and often have some form of operator input that is either active or passive. Similarly, imaging of travelers themselves to interrogate under clothing or the like is of a comparable type. In still other embodiments, cognitively demanding visual information may be selected without corresponding operator input and/or such input may be only passive. Such passive input may take the form of biometric information about the operator, operator eye movement detected with camera 37a, oral input with subsystem 35, and/or input with sensor(s) 39—just to name some nonlimiting examples.

From operation 324, procedure 320 continues with operation 326. In operation 326, test data appropriate to the platform selection in operation 324 is prepared for resource-based neuroenergetic analysis. In some cases, a sufficiently large dataset for the selected platform may be available while not for others—certainly not to the extent available for standardized testing previously described. As an addition or alternative, equipment-specific, operator-specific, group-specific, and/or other source(s) may be utilized to provide the desired degree of training data. In many such instances, machine learning may be used in lieu of or in concert with training data. Indeed, as experience with individual or group-sourced training data accumulates, greater confidence is likely to follow. Moreover, accumulated data may be organized to provide for training and proficiency testing of operators, among other things.

Procedure 320 continues with operation 328. In operation 328, routine 120 is executed with the corresponding data set to the extent available. Operation 328 is adjusted as needed to provide a suitable result when machine learned data, operator/equipment training sets, and the like are used alone or to augment other more readily available data, such as that obtained from global operations, manufacturers, software providers, or the like. It should be recognized that depending on the amount of trustworthy data, the outcome of operation 328 may be more limited than situations for which a large, high-confidence data set is available. Nonetheless, operation 328 is performed to preferably provide a useful resource-based neuroenergetic model, and more preferably a multiple resource neuroenergetic model. Even so, in other embodiments a different neuroenergetic model may be utilized or may be absent.

From operation 328, procedure 320 advances to operation 330. In operation 330, a cognitively demanding/intensive activity is performed by the operator (subject 22) with the platform selected in operation 324. Correspondingly, data prepared and collected from one or more sources or methodologies are assembled in operation 326 for application in operation 328 in any of the manners/modes previously described. While not always present, active operator input using keyboard 34, voice/sound subsystem 35, pointing device 36, and/or one or more biometric or other passive inputs like eye/facial movement detected with camera 37a, operator body information collected with sensor(s) 39, or the like is also acquired in operation 330. Nonetheless, in other embodiments, there may be little or no operator input provided. Alternatively or additionally, audio input to the operator (subject 22) can be used to provide the operator instructional/performance feedback and/or may be the subject of cognitively demanding analysis instead of visual information. Based on performance of a cognitively demanding visual activity (and/or a cognitively intensive activity of an aural variety), the operator provides the result(s) of the activity as input(s) or entries into equipment 31 using any of the aforementioned approaches previously described. It should be appreciated that in certain embodiments, such approaches are exclusive to verbal input via subsystem 35 without supplementation by any other modalities. In still other embodiments, cognitively demanding/intensive visual activity may be performed without providing any form of operator input of either an active or passive variety before concluding operation 330.

From operation 330, procedure 320 proceeds to operation 332 in which the cognitively demanding/intensive activity of the operator is monitored by evaluating any operator-generated result(s). As in the case of standardized testing, this evaluation includes determination of operator the timing and accuracy to the extent facilitated by available data. Operation 332 includes evaluation of operator input(s) relative to data representative of neuroenergetic modeling of the cognitively demanding/intensive activity performed by the operator. This evaluation may take the form of a comparison between operator input(s) and the data representing the model that may or may not subsume the determination of operator timing and/or accuracy. It should be appreciated that procedure 320 may introduce some or all of the visual information reviewed by the operator to test operator acuity, attention to the activity, understanding, training, fatigue, etc. . . . . For example, in the case of an application of X-ray security screening equipment for a traveler's carry-on belongings, the image stream presented to the operator may be altered to include one or more suspicious and/or even notably high-risk elements/features as part of the changing visual information ordinarily presented to the operator for review. The ability of the operator to identify such elements/features provides a way to evaluate the operator as an addition or alternative to neuroenergetic data application. Likewise, one or more elements/features may be omitted from an image stream to further test operator performance; where such elements/features should be readily apparent to a properly trained, rested, and attentive operator.

From operation 332, procedure 320 advances to conditional 340. Conditional 340 tests whether the most recent cognitively demanding/intensive activity performed by the operator, as evaluated in operation 332, provides an acceptable outcome. If the test of conditional 340 is negative (No), procedure 320 continues with operation 342. Operation 342 adjusts and repeats some or all of operations 326, 328, 330, and/or operation 332 (including some or all sub-operations thereof) (not shown), as estimated to provide a more acceptable outcome at the end of operation 332. After execution, operation 342 returns to conditional 340 to repeat the test thereof until the test result is affirmative (Yes). Next, procedure 320 moves from conditional 340 to conditional 350. Conditional 350 tests whether to continue procedure 320 or not. If the test of conditional 350 is affirmative (Yes), procedure 320 loops back to operation 324 to re-perform platform selection, prepare corresponding test data (operation 326), and re-perform operation 328. If the same platform, test data, and data modeling is to be used, conditional 350 need not loop back as far, restarting with operation 330. Procedure 320 continues with execution of operation 332, conditional 340 and operation 342, ultimately returning to conditional 350 and re-looping back to operation 324 until the test of conditional 350 is negative (No) at which point procedure 320 halts with stop stage 352.

Numerous additional embodiments are envisioned. For example, in certain embodiments, procedure 320 is tailored to a particular platform type or platform grouping with corresponding alteration to its operations and conditionals. In another example, procedure 320 is streamlined to rely on passive feedback from the operator (such as changes in eye movement or the like) to perform much, if not all, operator assessments. In a further example, a method comprises: receiving data input from an operator, the data input being representative of an evaluation of varying visual information included in a cognitively demanding activity performed by the operator; analyzing the data input from the operator relative to a data representation of a corresponding multiple resource neuroenergetic model of the cognitively demanding activity; in response to the analyzing of the data input from the operator, generating one or more operator instructions; and directing a break period for the operator from the cognitively demanding activity in accordance with the one or more operator instructions. Still another embodiment is directed to a method including: delivering information to computer equipment of a recipient; receiving an evaluation of the information conducted in performance of a cognitively intensive activity by the recipient; performing an assessment of the evaluation relative to data representative of a resource-based neuroenergetic model of the cognitively intensive activity; generating one or more outputs as a function of the assessment to manage operation of the cognitively intensive activity of the recipient through the computer equipment; and in response to the one or more outputs, managing the operation of the cognitively intensive activity by the recipient in accordance with the assessment. Yet another method is directed to a method that includes: reviewing a changing visual source of information; performing a cognitively demanding activity in connection with the reviewing of the changing visual source of information; providing one or more inputs responsive to the performing of the cognitively demanding activity; evaluating timing from the one or more inputs to produce representative input information; comparing the representative input information to data relating to a resource-based neuroenergetic model; and in response to the comparing of the representative input information, generating one or more outputs to manage the cognitively demanding activity. A further method includes: obtaining computer equipment entries from an operator in response to operator performance of a cognitively demanding activity, the cognitively demanding activity including evaluation of variable visual information by the operator; assessing data representative of a resource-based neuroenergetic model relative to the computer equipment entries of the operator; generating one or more results responsive to the assessing of the data, the one or more results corresponding to management of the cognitively demanding activity; and changing the visual information in response to the one or more results. Still a further method comprises: initiating performance of a cognitively demanding activity performed by an operator in response to varying visual information presented to the operator; attaining results generated by the operator in response to the cognitively demanding activity; analyzing the results to determine one or more of timing and accuracy; determining the one or more of the timing and the accuracy indicate resource depletion based of data representative of a resource-based neuroenergetic model of the cognitively demanding activity; and generating an output to address the resource depletion. Even further embodiments include systems to perform the above-indicated methods.

Another embodiment is directed to a system, comprising: means for receiving data input from an operator, means for analyzing the data input from the operator relative to a data representation of a corresponding multiple resource neuroenergetic model of the cognitively demanding activity; means for generating one or more operator instructions in response to the analyzing means; and means for directing a break period for the operator from the cognitively demanding activity in accordance with the one or more operator instructions. Still another embodiment is directed to a system including: means for delivering information to computer equipment of a recipient; means for receiving an evaluation of the information conducted in performance of a cognitively intensive activity by the recipient; means for performing an assessment of the evaluation relative to data representative of a resource-based neuroenergetic model of the cognitively intensive activity; means or generating one or more outputs as a function of the assessment to manage operation of the cognitively intensive activity of the recipient through the computer equipment; and means for managing the operation of the cognitively intensive activity by the recipient in accordance with the assessment in response to the one or more outputs. Yet another system includes: means for reviewing a changing visual source of information; means for performing a cognitively demanding activity in connection with the reviewing of the changing visual source of information; means for providing one or more inputs responsive to the performing of the cognitively demanding activity; means for evaluating timing from the one or more inputs to produce representative input information; means for comparing the representative input information to data relating to a resource-based neuroenergetic model; and means for generating one or more outputs to manage the cognitively demanding activity in response to the comparing means. A further system pertain to a system, comprising: means for obtaining computer equipment entries from an operator in response to operator performance of a cognitively demanding activity, the cognitively demanding activity including evaluation of variable visual information by the operator; means for assessing data representative of a resource-based neuroenergetic model relative to the computer equipment entries of the operator; means for generating one or more results responsive to the assessing of the data, the one or more results corresponding to management of the cognitively demanding activity; and means for changing the visual information in response to the one or more results. Still a further system comprises: means for initiating performance of a cognitively demanding activity performed by an operator in response to varying visual information presented to the operator; attaining results generated by the operator in response to the cognitively demanding activity; means for analyzing the results to determine one or more of timing and accuracy; and means for determining the one or more of the timing and the accuracy indicate resource depletion based of data representative of a resource-based neuroenergetic model of the cognitively demanding activity.

Any experiment, theory, thesis, hypothesis, mechanism, proof, example, belief, speculation, conjecture, guesswork, discovery, investigation, or finding stated herein is meant to further enhance understanding of the present application without limiting the construction or scope of any claim that follows or invention otherwise described herein—except to the extent expressly recited in such claim or invention. For any particular reference to “embodiment” or the like, any aspect(s) described in connection with such reference are included therein, but are not included in nor excluded from any other embodiment absent description to the contrary. For multiple references to “embodiment” or the like, some or all of such references refer to the same embodiment or to two or more different embodiments depending on corresponding modifier(s) or qualifier(s), surrounding context, and/or related description of any aspect(s) thereof—understanding two embodiments differ only if there is some substantive distinction, including but not limited to any substantive aspect described for one but not included in the other.

Any use of the words: important, critical, crucial, significant, essential, salient, specific, specifically, imperative, substantial, extraordinary, especially, favor, favored, favorably, favorable, desire, desired, desirable, desirably, particular, particularly, prefer, preferable, preferably, preference, and preferred indicates that the described aspects being modified thereby may be desirable (but not necessarily the only or most desirable), and further may indicate different degrees of desirability among different described aspects; however, the claims that follow are not intended to require such aspects or different degrees associated therewith except to the extent expressly recited.

For any method or process claim that recites multiple acts, conditionals, elements, gerunds, stages, steps, operations, phases, procedures, routines, and/or other claimed features; no particular order or sequence of performance of such features is thereby intended unless expressly indicated to the contrary as further explained hereafter. There is no intention that method claim scope (including order/sequence) be qualified, restricted, confined, limited, or otherwise influenced because: (a) the method/process claim as written merely recites one feature before or after another; (b) an indefinite article accompanies a method claim feature when first introduced and a definite article thereafter (or equivalent for method claim gerunds) absent compelling claim construction reasons in addition; or (c) the claim includes alphabetical, cardinal number, or roman numeral labeling to improve readability, organization, or other purposes without any express indication such labeling intends to impose a particular order. In contrast, to the extent there is an intention to limit a method/process claim to a particular order or sequence of performance: (a) ordinal numbers (1st, 2nd, 3rd, and so on) or corresponding words (first, second, third, and so on) shall be expressly used to specify the intended order/sequence; and/or (b) when an earlier listed feature is referenced by a later listed feature and a relationship between them is of such a type that imposes a relative order because construing otherwise would be irrational and/or any compelling applicable claim construction principle(s) support an order of the earlier feature before the later feature. However, to the extent claim construction imposes that one feature be performed before another, the mere ordering of those two features through such construction is not intended to serve as a rationale or otherwise impose an order on any other features listed before, after, or between them.

Moreover, no claim is intended to be construed as including a means or step for performing a specified function unless expressly introduced in the claim by the language “means for” or “step for,” respectively. As used herein, “portion” means a part of the whole, broadly including both the state of being separate from the whole and the state of being integrated/integral/contiguous with the whole, unless expressly stated to the contrary. Representative embodiments in the foregoing description and other information in the present application possibly may appear under one or more different headings/subheadings. Such headings/subheadings go to the form of the application only, which while perhaps aiding the reader, are not intended to limit scope or meaning of any embodiments, inventions, or description set forth herein, including any claims that follow. Only representative embodiments have been described, such that: advantages, apparatus, applications, arrangements, aspects, attributes, benefits, characterizations, combinations, components, compositions, compounds, conditions, configurations, constituents, designs, details, determinations, devices, discoveries, elements, embodiments, examples, exchanges, experiments, explanations, expressions, factors, features, forms, formulae, gains, implementations, innovations, kits, layouts, machinery, materials, mechanisms, methods, modes, models, objects, options, operations, parts, processes, properties, qualities, refinements, relationships, representations, species, structures, substitutions, systems, techniques, traits, uses, utilities, and/or variations that come within the spirit, scope, and/or meaning of any inventions defined and/or described herein, including any of the following claims, are desired to be protected.

All references listed herein are hereby incorporated by reference as if each were fully set forth in its entirety herein, including, but not limited to references listed in the corresponding U.S. Provisional Patent Application No. 62/193,409 filed 16 Jul. 2016.

1. A method, comprising:
receiving data input from an operator, the data input being representative of an evaluation of varying visual information included in a cognitively demanding activity performed by the operator;
analyzing the data input from the operator relative to a data representation of a corresponding multiple resource neuroenergetic model of the cognitively demanding activity;
in response to the analyzing of the data input from the operator, generating one or more operator instructions; and
directing a break period for the operator from the cognitively demanding activity in accordance with the one or more operator instructions.

2. The method of claim 1, in which:
the cognitively demanding activity corresponds to taking a standardized test;
the varying visual information corresponds to standardized test questions; and
the data input represents responses to the standardized test questions.

3. The method of claim 1, which includes:
predicting a break time from the analyzing of the data input relative to the data representation of the multiple resource neuroenergetic model; and
performing the directing of the break period in accordance with the predicting of the break time.

4. The method of claim 1, which includes:
operating computer equipment;
presenting the varying visual information to the operator with a display responsive to the computer equipment; and
the operator providing the data input through one or more operator input devices operatively coupled to the computer equipment.

5. The method of claim 4, in which the computer equipment belongs to at least one of: an air traffic control system, a security screening imaging system, a weapons system, a satellite imagery system, and a medical imaging system.

6. The method of claim 4, in which:
the computer equipment is responsive to one or more of a sound detection subsystem, a body sensor, a camera, a touchscreen input, a keyboard, and a pointing device; and
at least one of a display and an audio output subsystem is responsive to the computer equipment.

7. A method, comprising:
delivering information to computer equipment of a recipient;
receiving an evaluation of the information conducted in performance of a cognitively intensive activity by the recipient;
performing an assessment of the evaluation relative to data representative of a resource-based neuroenergetic model of the cognitively intensive activity;
generating one or more outputs as a function of the assessment to manage operation of the cognitively intensive activity of the recipient through the computer equipment; and
in response to the one or more outputs, managing the operation of the cognitively intensive activity by the recipient in accordance with the assessment.

8. The method of claim 7, in which the one or more outputs define a break time of the recipient from the cognitively intensive activity.

9. The method of claim 7, in which the resource=based neuroenergetic model is of a multiple resource type.

10. The method of claim 7, in which the neuroenergetic model is based on brain chemistry restoration and reduction of glucose and lactate.

11. The method of claim 7, in which the information defines a varying visualization with a display responsive to the computer equipment.

12. The method of claim 7, in which the computer equipment belongs to at least one of: an air traffic control system, a security screening imaging system, a weapons system, a satellite imagery system, and a medical imaging system.

13. The method of claim 7, in which:
the computer equipment is responsive to one or more of a sound detection subsystem, a body sensor, a camera, a touchscreen input, a keyboard, and a pointing device; and
at least one of a display and an audio output subsystem is responsive to the computer equipment.

14. A method, comprising:
reviewing a changing visual source of information;
performing a cognitively demanding activity in connection with the reviewing of the changing visual source of information;
providing one or more inputs responsive to the performing of the cognitively demanding activity;
evaluating timing from the one or more inputs to produce representative input information;
comparing the representative input information to data relating to a resource-based neuroenergetic model; and
in response to the comparing of the representative input information, generating one or more outputs to manage the cognitively demanding activity.

15. The method of claim 14, which includes evaluating accuracy between from the one or more inputs to produce further representative input information.

16. The method of claim 14, which includes presenting the changing visual source of information with a display responsive to computer equipment.

17. The method of claim 16, which includes at least one of a keyboard, a touchscreen input, a pointing device, a camera, a sound input subsystem, and a body sensor operatively coupled to the computer equipment.

18. The method of claim 16, in which the computer equipment belongs to at least one of: an air traffic control system, a security screening imaging system, a weapons system, a satellite imagery system, and a medical imaging system.

19. The method of claim 16, in which the resource-based neuroenergetic model is at least based on a restoration rate of glucose and a reduction rate of glucose.

20. A method, comprising:
obtaining computer equipment entries from an operator in response to operator performance of a cognitively demanding activity, the cognitively demanding activity including evaluation of variable visual information by the operator;
assessing data representative of a resource-based neuroenergetic model relative to the computer equipment entries of the operator;
generating one or more results responsive to the assessing of the data, the one or more results corresponding to management of the cognitively demanding activity; and
changing the visual information in response to the one or more results.

21. The method of claim 20, in which the resource-based neuroenergetic model is at least based on a restoration rate of glucose and a reduction rate of glucose.

22. The method of claim 20, which includes establishing a break time for the operator from the cognitively demanding activity.

23. The method of claim 20, which includes at least one of a keyboard, a touchscreen input, a pointing device, a camera, a sound input subsystem, and a body sensor operatively coupled to computer equipment.

24. The method of claim 23, in which the computer equipment belongs to at least one of: an air traffic control system, a security screening imaging system, a weapons system, a satellite imagery system, and a medical imaging system.
